(19) United States
(12) Patent Application Publication
(10) Pub. No.: US 2024/0165461 A1
(43) Pub. Date: May 23, 2024

(54) BREATH TRAINING APPARATUS

(71) Applicant: Eduard Johannis Adrianus Reuvers, Surin (TH)

(72) Inventor: Eduard Johannis Adrianus Reuvers, Surin (TH)

(21) Appl. No.: 18/282,793

(22) PCT Filed: Mar. 19, 2022
(86) PCT No.: PCT/IB2022/052512

§ 371 Date: Sep. 19, 2023

(51) Int. Cl. A63B 23/18; A61G 10/04; A61M 16/06

(52) U.S. Cl. CPC A63B 23/18; A61G 10/04; A61M 16/06; A61M 2202/0208; A61M 2202/0225; A63B 2230/438

(57) ABSTRACT

Described herein is a breath training apparatus [100], comprising a breathing unit [102], a CO₂ modulation unit [106, 108], and a control unit [112]. The breathing unit [102] has an enrichment airflow pathway [102c], and is adapted to provide inhalation air with enriched CO₂ concentration. The control unit [112] comprises a CO₂ actuator [112a, 112b], a CO₂ sensor [112d], and a controller [112g]. The controller: receives the signals corresponding to CO₂ concentration within the enrichment airflow pathway [102c] from the CO₂ sensor [112d], compares the CO₂ concentration with a defined CO₂ concentration; and correspondingly controls the CO₂ actuator [112a, 112b] for controlled manipulation of the CO₂ modulation unit [106, 108], such that the controlled manipulation of the at least one CO₂ modulation unit [106, 108] adjust the CO₂ concentration within the enrichment airflow pathway [102c] at least substantially equivalent to the defined CO₂ concentration.

### TECHNICAL FIELD

The present disclosure relates to a smart breath training apparatus. More particularly, the present invention relates to a control unit for controllably operating a breathing unit of the breath training apparatus.

### BACKGROUND

This section is intended to provide information relating to the field of the invention and thus any approach or functionality described below should not be assumed to be qualified as prior art merely by its inclusion in this section.

Various diseases are known these days, for example, migraine, epilepsy, febrile seizures, asthma, vascular disease, COPD, headache, sleep apnea, pneumonia, which can be treated with breath trainings/exercises. One example for treating the aforementioned diseases is by hyper-capnic breath training/exercise. Such hyper-capnic breath trainings/exercises include supplying inhalation air with the varied amount of carbon dioxide (CO₂) concentration and the varied amount of oxygen (O₂) concentration. It is very commonly known that such breath trainings/exercises, by supplying inhalation air with the varied amount of CO₂ concentration and the varied amount of O₂ concentration, imparts therapeutic effects to the user, particularly, while treating aforementioned diseases. Alternatively, such breath trainings/exercises of supplying inhalation air with the varied amount of carbon dioxide (CO₂) concentration and the varied amount of oxygen (O₂) concentration are also used by athletes and health & fitness enthusiasts for imparting training effects of increased physical stamina, physical strength, energy, mental focus, relaxation and the like. A breath training apparatus is commonly known to enable such breath trainings/exercises, i.e. to supply inhalation air with the varied amount of CO₂ concentration and the varied amount of O₂ concentration.

The breath training apparatus supplies inhalation air, wherein the CO₂ concentration and the O₂ concentration within the inhalation air can be adjusted. Commonly, the breath training apparatus is similar to a ventilator system. The breath training apparatus comprises of an O₂ supply unit, a CO₂ supply unit, a mixing unit fluidly connected to each of the O₂ supply unit and the CO₂ supply unit, a mask unit fluidly connected to the mixing unit. The mixing unit receives a controlled amount of O₂ from the O₂ supply unit, receives a controlled amount of CO₂ from CO₂ supply unit, and receives an amount of ambient air from external environment through small orifices. The mixing unit further mixes the controlled amount of O₂ concentration, the controlled amount of CO₂ concentration, and the amount of ambient air, to provide the mixture as inhalation air. Notably, the ambient air is received within the mixing unit through the small orifices, due to negative pressure generated by inhalation of the user. However, it may be noted that the ambient air may also include defined amount of CO₂ concentration and the O₂ concentration, and accordingly the actual CO₂ concentration and the actual O₂ concentration of the inhalation air is different from the user-defined concentration of CO₂ and the user-defined concentration of O₂ supplied by the CO₂ supply unit and the O₂ supply unit, respectively. In such conventionally known breath training apparatuses, it is difficult for a user to refill and replenish the CO₂ supply unit with fresh CO₂. Additionally, in such conventional breath training apparatuses, as the ambient air is received through small orifices positioned at the air inlet, it increases an air resistance while breathing, which forces diaphragm of user to function in an unnaturally forceful way, leading to poor simulation of actual climatic conditions. Moreover, in the conventionally known breath training apparatuses, it is difficult for the user to achieve precise control of the CO₂ concentration and the O₂ concentration within the inhalation air.

Another example of the breath training apparatus includes a rebreathing device that may be used to provide an ongoing supply of inhalation air with an increased amount of CO₂ concentration and a low amount of O₂ concentration, without use of pressurized tanks (CO₂ supply unit and O₂ supply unit). Many of these rebreathing devices are not able to provide inhalation air at a sufficiently increased amount of at least 5% CO₂ concentration (as used in the common form of Carbogen air mixture) or the increased amount of at least 8% CO₂ concentration for increased level training by athletes and health & fitness enthusiasts. These rebreathing devices commonly also provide extra airflow resistance to the airflow of user, either as an unintended side-effect or as a therapeutic or training effect, for example by forcing the breathing airflow of user through a narrow air passage or through pipes which contain some form of moveable or variable obstruction, or by employing an air mixing unit using small airflow orifices, or by receiving the users exhalation airflow into an expandable bag or balloon. All these provide a noticeable resistive effect on the smoothness of the users breathing airflow, which forces the diaphragm of user to function in an unnaturally forceful way. Also, the rebreathing devices commonly use a bag or a closed compartment with flexible walls or a fixed compartment with small orifices to contain the exhaled air, which is then provided back to the user in the next inhalation. These bags or compartments become very moist inside after a few minutes, which promotes bacterial growth and are difficult to dry out and be cleaned due to their closed nature.

Moreover, in the conventionally known breath training apparatuses, it is difficult for the user to achieve precise control of the CO₂ concentration and the O₂ concentration within the inhalation air, where there is no need to connect a pressurized tank containing either CO₂ or O₂, where the breath training apparatus also is capable of providing inhalation air with substantially high level of CO₂ concentration, and wherein the breath training apparatus minimizes extra airflow resistance in the breathing experience of the user and prevents bacterial growth within the apparatus by drying out quickly and naturally after use and where the apparatus is also very easy to clean.

PCT patent numbered WO 2006/107117 A1 (hereinafter referred to as WO'117 patent application), discloses a rebreathing device for increasing the level of CO₂ concentration in the inhalation air by using a simple pipe shape. Although, it is disclosed that the rebreathing device minimizes extra airflow resistance to the breathing airflow giving the user a relatively natural breathing experience, however the device is unable to reach high levels of CO₂ concentration in the inhalation air, due to its low capacities. The device also fails to disclose any method to precisely determine the CO₂ concentration and/or the O₂ concentration within the inhalation air, and also fails to disclose any method to precisely control the CO₂ concentration and/or the O₂ concentration within the inhalation air.

United States Patent numbered US 2019/0351161 A1 (hereinafter referred to as US'161 patent application), discloses a breathing device for increasing the level of CO₂ (i.e. CO₂ concentration) in the inhalation air, using a flexible breathing chamber such as a bag. The US'161 patent application discloses a mouthpiece being configured for a user breathing into the mouthpiece through a breathing opening, and at least partly flexible rebreathing air chamber (preferably cuboidal shaped) fluidly connected to the mouthpiece, wherein the rebreathing air chamber has a first wall section being gas permeable through either of a plurality of pores or through going openings for fluid communication with surrounding atmosphere. Such breathing device employs exhaled air in exhalation cycle to be used as inhalation air in next inhalation cycle with increased CO₂ concentrations. Further, a CO₂ sensing unit is also provided to monitor CO₂ concentration in exhaled air and/or inhalation air, for raising an alarm as the CO₂ concentration reaches beyond acceptable limits. Although, the present disclosure discloses usage of at least a portion of exhaled air in one exhalation cycle to be used as inhalation air in next inhalation cycle for increased CO₂ concentration in the inhalation air, however, the present disclosure at least fails to disclose a control system for precisely controlling CO₂ concentration provided in the inhalation air. Moreover, as the present disclosure discloses usage of a bag as a flexible breathing chamber within which the user exhales and inhales, it results in imparting a relatively artificial breathing simulation to the user, thereby resulting in harder and unnatural breathing experience to the user. Additionally, such usage of such flexible breathing chamber employing a bag for the purpose, are prone to get moist quickly, resulting in increased bacteria growth therein, and is thus also relatively difficult to clean. The breathing device disclosed herein also fails to disclose any system/method to increase/vary the O2 concentration in the inhalation air.

European Patent numbered EP2338575 A1 (hereinafter referred to as EP'575 patent application), discloses a respiratory muscle endurance training (RMET) device that includes: a patient interface for transferring a patient's exhaled or inhaled gases; a fixed volume chamber in communication with the patient interface to retain a portion of a patient's exhaled gases; a variable volume chamber in communication with the fixed volume chamber to be responsive to the patient's exhaled or inhaled gases to move from a first position to a second position, wherein the variable volume chamber is a rebreathing bag, preferable of 1 to 2 l in volume; and a variable orifice adjustable for permitting a portion of exhaled air to escape to the ambient air during exhalation and receiving a supply of fresh air during inhalation. Thus, the device is capable of increasing CO₂ concentration therein. Additionally, the respiratory muscle endurance training (RMET) device employs a patient interface, wherein a CO₂ sensor coupled to a chamber to provide the user or caregiver with indicia about the CO₂ level. Although, the present disclosure discloses the device capable of providing inhalation air with increased CO₂ concentration, however, the present disclosure fails to disclose a control system for precisely controlling CO₂ concentration provided in the inhalation air. Moreover, as the present disclosure discloses usage of a rebreathing bag as a variable volume chamber within which the user exhales and/or inhales, it results in imparting a relatively artificial breathing simulation to the user, thereby resulting in harder and unnatural breathing experience to the user. Additionally, such usage of such variable volume chamber employing a bag for the purpose, are prone to get moist quickly, resulting in increased bacteria growth therein, and is thus also relatively difficult to clean. The RMET disclosed herein also fails to disclose any system/method to increase/vary the O2 concentration in the inhalation air.

In addition to aforementioned drawbacks of the breath training apparatuses, there is a well felt need of the breath training apparatus that can provide inhalation air with precise control of CO₂ concentration and the O₂ concentration within the inhalation air.

### SUMMARY

One aspect of the present disclosure relates to a breath training apparatus, comprising a breathing unit, at least one CO₂ modulation unit, and a control unit. The breathing unit defines an air inlet, an air outlet, and an enrichment airflow pathway therebetween. The breathing unit is adapted to: receive exhaled air from a user in at least one exhalation pass through the air inlet; enable a portion of air in the enrichment airflow pathway to vent through the air outlet, such that the remaining air in the enrichment airflow pathway mixes with the exhaled air to be enriched in CO₂ concentration, and release at least a portion of the enriched air through the air inlet for inhalation in subsequent inhalation pass to the user. The at least one CO₂ modulation unit is coupled to the breathing unit for modulating the CO₂ concentration in the enriched air, such that a controlled manipulation of the at least one CO₂ modulation unit corresponds to a control of CO₂ concentration within the enrichment airflow pathway of the breathing unit. The control unit comprises at least one CO₂ actuator, a CO₂ sensor, and a controller. The at least one CO₂ actuator controllably manipulates the at least one CO₂ modulation unit to control the CO₂ concentration within the enrichment airflow pathway of the breathing unit. The CO₂ sensor generates signals corresponding to CO₂ concentration of the enriched air within the enrichment airflow pathway of the breathing unit. The controller is adapted to: receive the CO₂ signals corresponding to CO₂ concentration within the enrichment airflow pathway from the CO₂ sensor, compare the CO₂ concentration with a defined CO₂ concentration; and correspondingly control the at least one CO₂ actuator for controlled manipulation of the at least one CO₂ modulation unit, such that the controlled manipulation of the at least one CO₂ modulation unit adjust the CO₂ concentration within the enrichment airflow pathway at least substantially equivalent to the defined CO₂ concentration.

Another object of the present disclosure relates to a method for automatically controlling a breath training apparatus. The method initiates with receiving, by an air inlet of a breathing unit, an exhaled air from a user in at least one exhalation pass. Thereafter, the method proceeds to enable, by the breathing unit, at least a portion of air in the enrichment airflow pathway to vent through the air outlet, such that the remaining air in the enrichment airflow pathway mixes with the exhaled air to be enriched in CO₂ concentration. Further, the method proceeds to release, by the air inlet of the breathing unit, at least a portion of the enriched air for inhalation in subsequent inhalation pass to the user. Thereafter, CO₂ signals are generated by a CO₂ sensor, corresponding to CO₂ concentration of the enriched air within the enrichment airflow pathway of the breathing unit. Upon generating the CO₂ signals, a controller receives the CO₂ signals. Thereafter, a defined CO₂ concentration from a user is received by the I/O unit. Thereafter, the controller compares the CO₂ concentration based on the CO₂ signals with the defined CO₂ concentration. Finally, the controller controls the at least one CO₂ actuator for controlled manipulation of the at least one CO₂ modulation unit, such that the controlled manipulation of the at least one CO₂ modulation unit adjust the CO₂ concentration within the enrichment airflow pathway at least substantially equivalent to the defined CO₂ concentration.

### BRIEF DESCRIPTION OF DRAWINGS

The present invention, both as to its organization and manner of operation, together with further objects and advantages, may best be understood by reference to the following description, taken in connection with the accompanying drawings. These and other details of the present invention will be described in connection with the accompanying drawings, which are furnished only by way of illustration and not in limitation of the invention, and in which drawings:

FIG. 1a shows a side sectional view of a breath training apparatus, illustrating various component of the breath training apparatus, in accordance with the concepts of a first embodiment the present disclosure.

FIG. 1b shows a perspective view of the sectional view of a breath training apparatus, illustrating various component of the breath training apparatus, in accordance with the concepts of a second embodiment the present disclosure.

FIG. 2 shows a perspective view of the breath training apparatus, illustrating a second CO₂ modulation unit in a retracted position, in accordance with the concepts of the first embodiment of the present disclosure.

FIG. 3 shows a perspective view of the breath training apparatus, illustrating a second CO₂ modulation unit in an extended position, in accordance with the concepts of the first embodiment of the present disclosure.

FIG. 4 shows a perspective view of a portion of the breath training apparatus, illustrating an O2 modulation unit, in accordance with the concepts of the first embodiment of the present disclosure.

FIG. 5 shows a perspective view of the breath training apparatus, illustrating an arrangement of a cocoon-type mask unit, in accordance with the concepts of a third embodiment of the present disclosure.

FIG. 6 shows a flowchart of a method of controlling the breathing unit of the breath training apparatus, in accordance with the concepts of the first embodiment of the present disclosure

### DETAILED DESCRIPTION

In the following description, for the purposes of explanation, various specific details are set forth in order to provide a thorough understanding of embodiments of the present invention. It will be apparent, however, that embodiments of the present invention may be practiced without these specific details. Several features described hereafter can each be used independently of one another or with any combination of other features. An individual feature may not address any of the problems discussed above or might address only one of the problems discussed above. Some of the problems discussed above might not be fully addressed by any of the features described herein. Example embodiments of the present invention are described below, as illustrated in various drawings in which like reference numerals refer to the same parts throughout the different drawings.

Unless otherwise stated, the terms “include” and “comprise” (and variations thereof such as “including”, “includes”, “comprising”, “comprises”, “comprised” and the like) are used inclusively and do not exclude further features, components, integers, steps or elements.

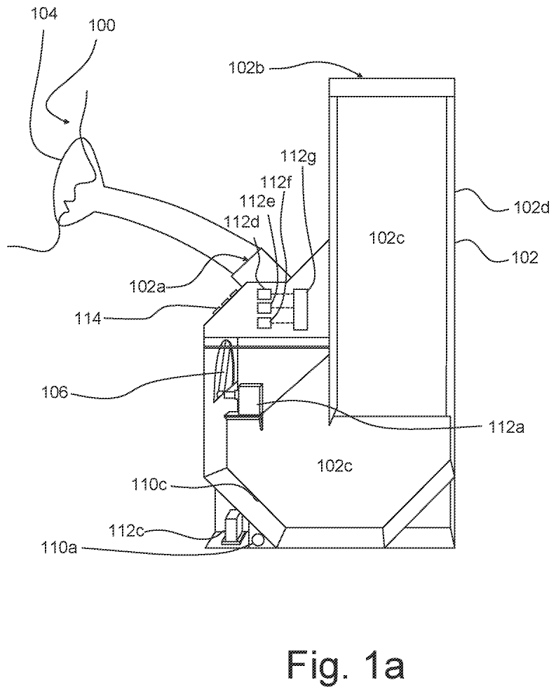

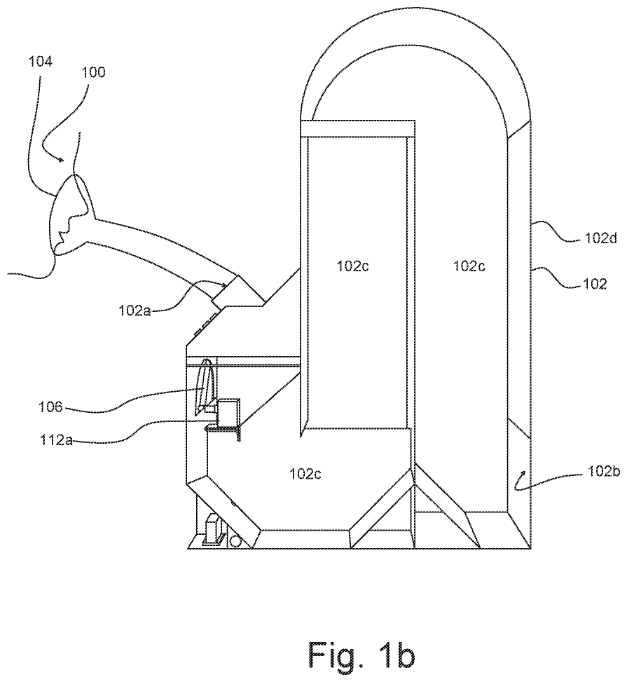

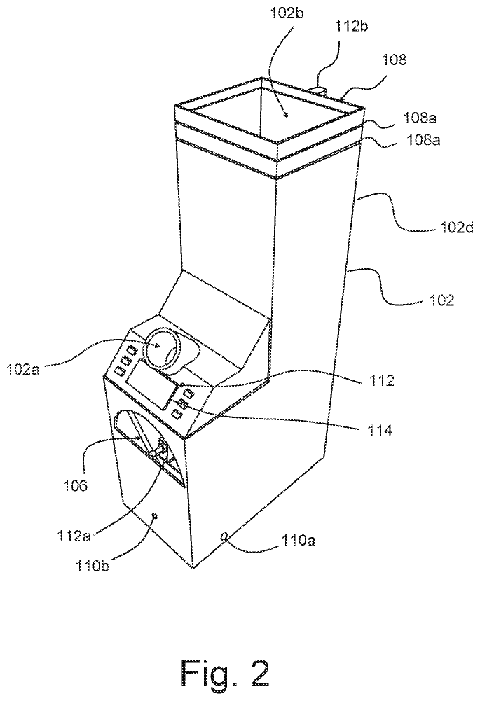

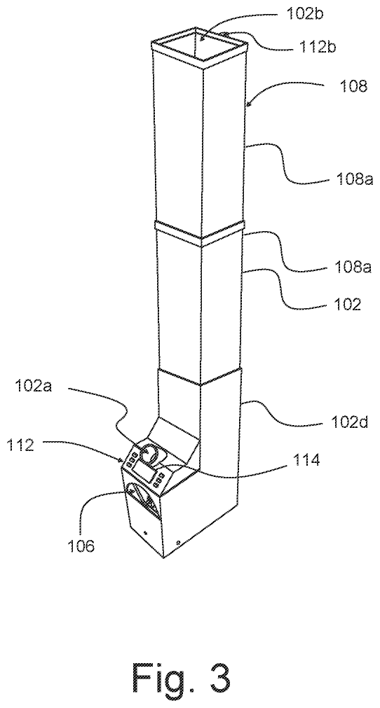

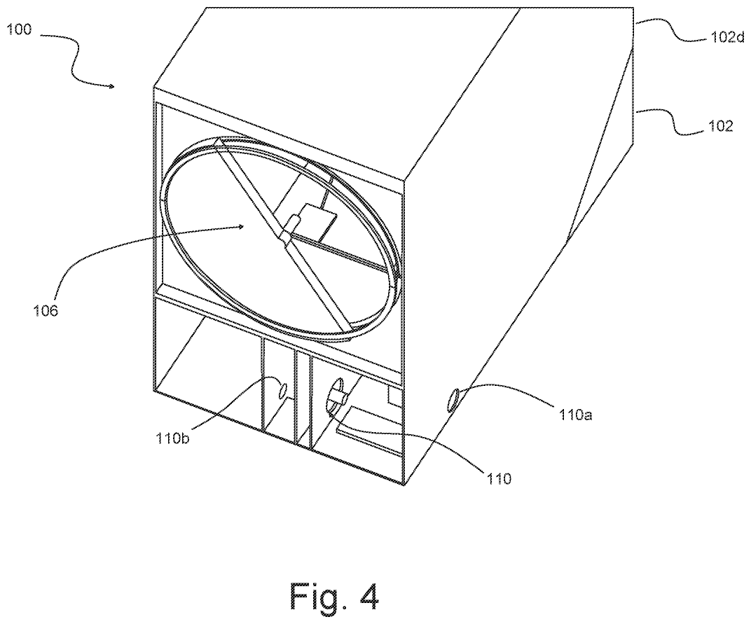

FIG. 1a shows a side sectional view of a breath training apparatus [100], illustrating various component of the breath training apparatus [100], in accordance with the concepts of the first embodiment of present disclosure. FIG. 2 shows a perspective view of the breath training apparatus [100], illustrating a second CO₂ modulation unit in a retracted position, in accordance with the concepts of the first embodiment of the present disclosure. FIG. 3 shows a perspective view of the breath training apparatus [100], illustrating a second CO₂ modulation unit in an extended position, in accordance with the concepts of the present disclosure. FIG. 4 shows a perspective view of a portion of the breath training apparatus [100], illustrating an O2 modulation unit, in accordance with the concepts of the first embodiment of the present disclosure. FIGS. 1a, 2, 3, and 4 should be referred to in conjunction with each other to clearly understand a scope of the present disclosure. The breath training apparatus [100] is adapted to provide inhalation air with increased CO₂ concentration while lowering, maintaining, or increasing the O₂ concentration, such that inhalation air comprises of a mixture of at least a portion of exhaled air and ambient air. As is understood to a person ordinarily skilled in the art that the O₂ concentration can be either lowered, maintained, or increased, concepts of the present disclosure hereinafter will be described as the O₂ concentration been increased along with increase of the CO₂ concentration. Moreover, the breath training apparatus [100] is adapted to provide inhalation air, wherein CO₂ concentration and O₂ concentration within the inhalation air can be precisely controlled, such that the breath training apparatus [100] can be efficiently used for breath training purposes. The breath training apparatus [100] includes a breathing unit [102], a mask unit [104], at least one CO₂ modulation unit [106 108], an O2 supply unit (not shown), an O₂ modulation unit [110], and a control unit [112].

The breathing unit [102] is fluidly connected to a mouth and/or nose of a user via the mask unit [104], and is suitably structured and arranged to perform the following: receive exhaled air from the user in at least one exhalation pass; enable a portion of air within the breathing unit to vent, such that the remaining air in the breathing unit [102] mixes with the exhaled air to be enriched in CO₂ concentration; and release at least a portion of the enriched air as inhalation air, for inhalation in subsequent inhalation pass to the user. As the enriched air is used for inhalation, the enriched air will be interchangeably referred to as inhalation air in forthcoming disclosure. A structure and arrangement of the breathing unit [102] for performing the aforementioned function will be described in details hereinafter. The breathing unit [102] includes at least partially enclosed housing unit [102d] that defines an air inlet [102a], an air outlet [102b], and an enrichment pathway [102c] defined between the air inlet [102a] and the air outlet [102b]. The enrichment pathway [102c] has a defined trajectory.

In a first embodiment (not shown) of the breathing unit [102], the housing unit [102d] is a cylindrical housing unit that defines the air inlet [102a] and the air outlet [102b] at opposite ends, and defines the enrichment airflow pathway [102c] with a straight trajectory defined between the air inlet [102a] and the air outlet [102b]. In a second embodiment of the breathing unit [102], as is shown in FIG. 1a, the housing unit [102d] is an enclosed cuboidal structure supporting at least one baffle plate structures therein, such that the air inlet [102a] and the air outlet [102b] are defined at two open ends, while the enrichment airflow pathway [102c] has a curved trajectory defining one (1) turn between the air inlet [102a] and the air outlet [102b]. In this embodiment, the enrichment airflow pathway [102c] may include one turn in the curved trajectory, defining two airflow pathways fluidly connected to each other. In yet another third embodiment of the breathing unit [102], as is shown in FIG. 1b, the housing unit [102d] is an enclosed structure supporting at least one baffle plate structures therein, such that the air inlet [102a] and the air outlet [102b] are defined at two open ends, while the enrichment airflow pathway [102c] has a curved trajectory defining two turns between the air inlet [102a] and the air outlet [102a]. In yet another embodiment of the breathing unit [102], the housing unit [102d] is an enclosed structure supporting two baffle plate structures therein, such that the air inlet [102a] and the air outlet [102b] are defined at two open ends, while the enrichment airflow pathway [102c] has a curved trajectory defining five turns between the air inlet [102a] and the air outlet [102b]. Each of the aforementioned embodiments are within a scope of the present disclosure, and is not described in details herein for the sake of brevity. It may be appreciated by a person ordinarily skilled in the art that the number of turns defined by the curved trajectory between the air inlet [102a] and the air outlet [102b], does not limit a scope of the invention, and any number of turns can be defined by the curved trajectory between the air inlet [102a] and the air outlet [102b]. The embodiment of the breathing unit [102], as shown in FIG. 1a, wherein the housing unit [102d] is structured and arranged, such that the enrichment airflow pathway [102c] includes the curved trajectory with one turn, is shown and described in details in the present disclosure.

In the preferred second embodiment of the breathing unit [102], the housing unit [102d] is a cuboidal structure that supports a baffle plate structure therein, such that the enrichment airflow pathway [102c] defines the one (1) turn therein, and is entirely defined between the air inlet [102a] and the air outlet [102b]. It may be noted that the housing unit [102d] is suitably structured, such that the enrichment airflow pathway [102c] has a volume (or capacity) in a range of 2.5 to 35 liters, preferably in a range of 5 to 15 liters. Such a volume (or capacity) of the enrichment airflow pathway [102c] is equal and/or significantly above a vital capacity of an average user to achieve higher level of CO₂ concentration in the inhalation air. The breathing unit [102] is capable of imparting the breath training to the user without causing much air resistance during the training process. Additionally, to achieve low airflow resistance and a high level of CO₂ concentration within the inhalation air, at least a width of the enrichment airflow pathway [102c] in a range of 6 cm to 17 cm, preferably in a range of 9 cm to 12.5 cm.

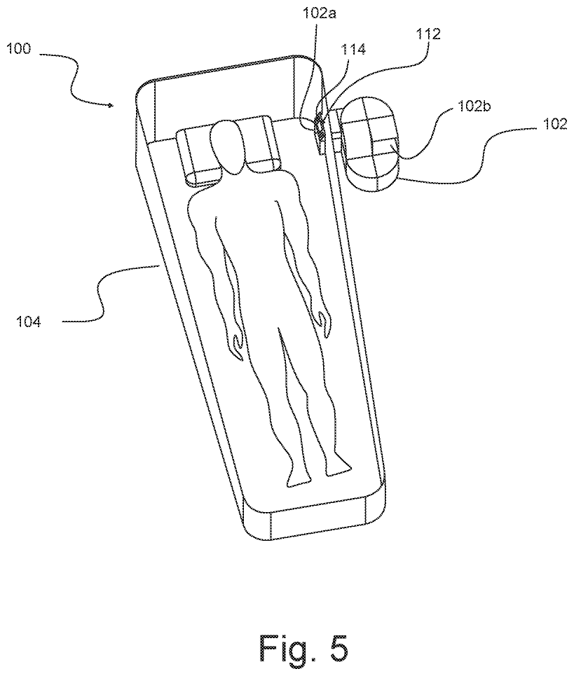

The mask unit [104] is installed over at least a portion of mouth and/or nose of user and is fluidly connected to the air inlet [102a] of the breathing unit [102] to: transfer exhaled air from the user to the breathing unit [102] during exhalation passes, and transfer inhalation air from the breathing unit [102] to the user during inhalation passes. In one embodiment, the mask unit [104] is a conventional mask unit comprising of a mask covering mouth and/or nose of a user, and a fluid connection pipe in communication with the mask and also fluidly connected to the air inlet [102a] of the breathing unit [102]. Thus, the embodiment of conventional mask unit [104] enables: transfer of exhaled air from the user to the breathing unit [102] during exhalation passes, and transfer of inhalation air from the breathing unit [102] to the user during inhalation passes. In another embodiment, as shown in FIG. 5, the mask unit [104] is a tent unit or a cocoon unit [104] with at least one opening, and a pipe connection in fluid communication with the tent unit or a cocoon unit and also fluidly connected to the air inlet [102a] of the breathing unit [102]. Thus, the embodiment of tent-type mask unit [104] enables: transfer of exhaled air from the user to the breathing unit [102] during exhalation passes, and transfer of inhalation air from the breathing unit [102] to the user during inhalation passes. In yet another embodiment, the mask unit [104] is a helmet-type unit housing at least a head of a user, in fluid communication with the air inlet [102a] of the breathing unit [102]. Thus, the embodiment of helmet-type mask unit [104] enables: transfer of exhaled air from the user to the breathing unit [102] during exhalation passes, and transfer of inhalation air from the breathing unit [102] to the user during inhalation passes.

In applications, the breathing unit [102] is adapted to: receive exhaled air from a user in at least one exhalation pass through the air inlet [102a]; enable a portion of air in the enrichment airflow pathway [102c] to vent through the air outlet [102b], such that the remaining air in the enrichment airflow pathway [102c] mixes with the exhaled air to be enriched in CO₂ concentration; and release at least a portion of the enriched air through the air inlet [102a] for inhalation in subsequent inhalation pass to the user. In an exemplary embodiment, in a first exhalation pass, a user may exhale air to the enrichment airflow pathway [102c] through the air inlet [102a] of the breath training unit [102]. Such exhalation of air into the enrichment airflow pathway [102c] enables pushing of at least a portion of the air already present in the enrichment airflow pathway [102c] to vent through the air outlet [102b], to ambient environment, and the remaining air in the enrichment airflow pathway [102c] mixes with the exhaled air. Notably, a mixture of exhaled air and ambient air is guided along the enrichment airflow pathway [102c] as it flows back and forth due to the forces of natural airflow (subsequent inhalation and exhalation) caused by a breathing of the user. With such mixing of the exhaled air with the remaining portion of the air already present in the enrichment airflow pathway [102c], the air is enriched in the CO₂ concentration. For example, the enriched air has 1% CO₂ concentration. Thereafter, in a subsequent first inhalation pass, the enriched air is available as inhalation air containing 1% CO₂ concentration to be inhaled by the user. Further, in a second exhalation pass, the enriched air is further similarly enriched, to increase the CO₂ concentration, for example to 2% CO₂ concentration. Subsequently, in a second inhalation pass, the enriched air, for example with 2% CO₂ concentration, is available for inhalation to the user. It may be noted that the CO₂ concentration in the enriched air is increased to a maximum CO₂ concentration, depending on a maximum volume of the enrichment airflow pathway [102c], and accordingly, the maximum CO₂ concentration of the enriched air is kept within limits.

The at least one CO₂ modulation unit [106, 108] is installed to modulate the CO₂ concentration within the enriched air available as inhalation air in the enrichment airflow pathway [102c] of the breathing unit [102]. The at least one CO₂ modulation unit [106, 108] is coupled to the breathing unit for modulating the CO₂ concentration in the enriched air, such that a controlled manipulation of the at least one CO₂ modulation unit [106, 108] corresponds to a control of CO₂ concentration within the enrichment airflow pathway of the breathing unit [102]. In the present embodiment, the at least one CO₂ modulation unit [106, 108] includes two CO₂ modulation units, namely a first CO₂ modulation unit [106] and a second CO₂ modulation unit [108].

The first CO₂ modulation unit [106] is rotary valve with a closable opening defined in the housing unit [102d], positioned proximal to the air inlet [102a] of the breathing unit [102]. The first CO₂ modulation [106] enables a (additional) shorter controlled fluid communication of the enrichment airflow pathway [102c] with the ambient air in external environment, which enables the exhaled air to at least partially bypass the enrichment airflow pathway [102c]. Notably, an amount of opening defined by the rotary valve to the external environment, defines the amount of CO₂ concentration in the exhaled air disposed in the enrichment airflow pathway [102c] of the breathing unit [102]. Accordingly, the first CO₂ modulation unit can be controllably manipulated, by controlling the amount of opening defined by the rotary valve, for controlling the CO₂ concentration within the enrichment air disposed in the enrichment airflow pathway [102c] of the breathing unit [102]. Although, the present disclosure describes the first CO₂ modulation unit [106] as the rotary valve, it may be obvious to a person ordinarily skilled in the art that the first CO₂ modulation unit [106] may include any of a sliding valve, a door mechanism, and/or any other similar mechanism that enables controlled fluid communication of the enrichment airflow pathway [102c] with the ambient air in external environment.

The second CO₂ modulation unit is a combination of one or more secondary housing units [108a] telescopically extending from the housing unit [102d] of the breathing unit [102]. A controlled telescopic extension and/or retraction of the one or more secondary housing units [108a] relative to the housing unit [102d], enlarges and/or contracts the enrichment airflow pathway [102c], thereby decreasing/increasing the amount of CO₂ concentration in the exhaled air disposed in the enrichment airflow pathway [102c] of the breathing unit [102]. Accordingly, the second CO₂ modulation unit can be controllably manipulated, by controllably extending/retracting the one or more secondary housing units [108a], for controlling the CO₂ concentration within the enrichment air disposed in the enrichment airflow pathway [102c] of the breathing unit [102].

Furthermore, as the CO₂ concentration within the enrichment air (or inhalation air) is increased, the O₂ concentration within the enrichment air may be reduced. Accordingly, the O₂ concentration within the enrichment air is also required to be controlled by the breath training apparatus [100], disclosed herein. Therefore, the breath training apparatus [100] employs the O₂ supply unit and the O₂ modulation unit [110], for controlling the O₂ concentration within the inhalation air disposed in the enrichment airflow pathway [102c] of the breathing unit [102].

The O₂ supply unit is fluidly communicated with the enrichment airflow pathway [102c] of the breathing unit [102] through an O₂ connection point [110a], to supply O₂ within the enrichment airflow pathway [102c] of the breathing unit [102], for controllably enriching the enriched air with O₂ concentration. The O₂ supply unit is an oxygen concentrator or another type of oxygen supply system that supplies O₂ when required. It may be noted that the O₂ supply unit is an external supportive O₂ supply unit, and thus is interchangeably referred to as the O₂ supply unit and the external O₂ supply unit, hereinafter.

Furthermore, the O₂ modulation unit [110] is provided to control the O₂ concentration in the enriched air (or inhalation air) disposed in the enrichment airflow pathway [102c] of the breathing unit [102]. The O₂ modulation unit [110] is an O₂ rotary valve with a closeable opening defined within the fluid communication between the O₂ supply unit and the enrichment airflow pathway [102c] of the breathing unit [102], such that the O₂ modulation unit [110] vents a portion of the O₂ received from O₂ supply unit to external environment through an O₂ vent hole [110b]. Remaining portion of the O₂ received from O₂ supply unit is guided to the exhaled air disposed in the enrichment airflow pathway [102c] of the breathing unit through an O₂ entry hole [110c]. Notably, an amount of opening of the O₂ rotary valve to the external environment, defines the amount of O₂ concentration in the exhaled air disposed in the enrichment airflow pathway [102c] of the breathing unit [102]. Accordingly, the O₂ modulation unit [110] can be controllably manipulated, by controlling the amount of opening of O₂ rotary valve, for controlling the O₂ concentration within the enrichment air disposed in the enrichment airflow pathway [102c] of the breathing unit [102]. Although, the present disclosure describes the O₂ modulation unit [110] as the O₂ rotary valve, it may be obvious to a person ordinarily skilled in the art that the O₂ modulation unit [110] may include any of a sliding valve, a door mechanism, and/or any other similar mechanism that enables venting of a portion of the O₂ received from O₂ supply unit to external environment.

The control unit [112] is provided for precise control of the CO₂ concentration and the O₂ concentration in the enriched air in the enrichment airflow pathway [102c]. The control unit includes at least one CO₂ actuator [112a, 112b], an O₂ actuator [112c], a CO₂ sensor [112d], an O₂ sensor [112e], one or more physiological parameter sensor, at least one air parameter sensor [112f], an input/output (I/O unit) [114], and a controller [112g].

The at least one CO₂ actuator [112a, 112b] is provided to controllably manipulate the at least one CO₂ modulation unit, to control the CO₂ concentration within the enrichment airflow pathway of the breathing unit. The at least one CO₂ actuator [112a, 112b] includes a first CO₂ actuator [112a] and a second CO₂ actuator [112b]. The first CO₂ actuator [112a] is a motor coupled to the rotary valve of the first CO₂ modulation unit. The first CO₂ actuator [112a] is adapted to controllably manipulate the rotary valve and control the opening defined by the rotary valve in the first CO₂ modulation unit [106], for correspondingly controlling the CO₂ concentration within the enrichment airflow pathway [102c] of the breathing unit [102]. Examples of the motor may include, but is not limited to a stepper motor, an induction motor, a 2-phase motor, a 3-phase motor, a servo motor, and the like. The second CO₂ actuator [112b] includes one or more linearly extendable/retractable electrically actuated cylinders coupled to the one or more secondary housing units [108a] of the second CO₂ modulation unit [108]. The second CO₂ actuator [112b] is adapted to controllably manipulate the second CO₂ modulation unit [108], for controlled extension/retraction of the one or more secondary housing units [108a] of the second CO₂ modulation unit [108], for correspondingly controlling the CO₂ concentration within the enrichment airflow pathway [102c] of the breathing unit [102]. Although, the present disclosure describes the second CO₂ actuator [112b] for controllably manipulating the second CO₂ modulation unit [108], it may be obvious to a person ordinarily skilled in the art that the second CO₂ modulation unit [108] can also be manually manipulated.

The O₂ actuator [112c] is provided to controllably manipulate the at least one O₂ modulation unit [110], to control the O₂ concentration within the enrichment airflow pathway [102c] of the breathing unit [102]. The O₂ actuator [112c] is a motor coupled to the O₂ rotary valve of the O₂ modulation unit [110]. The O₂ actuator [112c] is adapted to controllably manipulate the O₂ rotary valve and control the opening defined by the O₂ rotary valve in the O₂ modulation unit [110], for correspondingly controlling the O₂ concentration within the enrichment airflow pathway [102c] of the breathing unit [102]. Examples of the motor may include, but is not limited to a stepper motor, an induction motor, a 2-phase motor, a 3-phase motor, a servo motor, and the like.

The CO₂ sensor [112d] is provided to generate signals corresponding to CO₂ concentration of the enriched air within the enrichment airflow pathway [102c] of the breathing unit [102]. In one embodiment, the CO₂ sensor [112d] is a CO₂ level sensor that directly determines the CO₂ concentration in the enriched air disposed within the enrichment airflow pathway [102c] of the breathing unit [102]. In the embodiment of the CO₂ sensor [112d] deployed as the CO₂ level sensor, the CO₂ sensor [112d] includes an air receiving tube positioned opposite to a direction of inhalation of the enriched air for receiving at least a portion of the enriched inhalation air, to further determine the CO₂ concentration in the enriched air inhaled during inhalation pass. In another embodiment, the CO₂ sensor [112d] is either of an air pressure sensor and an air flow sensor that determines the air pressure and the air flow, respectively, of the enriched air disposed within the enrichment airflow pathway [102c], to indirectly determine the CO₂ concentration in the enriched air disposed within the enrichment airflow pathway [102c] of the breathing unit [102]. Further, the CO₂ sensor [112d] is adapted to generate a signal corresponding to the CO₂ concentration in the enriched air disposed within the enrichment airflow pathway [102c] of the breathing unit [102]. For example, a strength of the signal generated by the CO₂ sensor [112d] determines the CO₂ concentration in the enriched air disposed within the enrichment airflow pathway [102c] of the breathing unit [102].

Similar to the CO₂ sensor [112d], the O₂ sensor [112e] is provided to generate signals corresponding to O₂ concentration of the enriched air within the enrichment airflow pathway [102c] of the breathing unit [102]. The O₂ sensor [112e] is an O₂ level sensor that directly determines the CO₂ concentration in the enriched air disposed within the enrichment airflow pathway [102c] of the breathing unit [102]. The O₂ sensor [112e] includes an air receiving tube positioned opposite to a direction of inhalation of the enriched air for receiving at least a portion of the enriched inhalation air, to further determine the O₂ concentration in the enriched air inhaled during inhalation pass. Further, the O₂ sensor [112e] is adapted to generate a signal corresponding to the O₂ concentration in the enriched air disposed within the enrichment airflow pathway [102c] of the breathing unit [102]. For example, a strength of the signal generated by the O₂ sensor [112e] determines the O₂ concentration in the enriched air disposed within the enrichment airflow pathway [102c] of the breathing unit [102].

The one or more real-time physiological sensors is connected to a user, and is adapted to determine one or more real-time physiological parameters related to a user. Real-time refers to actual values at point of time. The real-time physiological parameter of the user may include, but is not limited to, a real-time heart rate, a real-time exhaled airflow pressure, a real-time exhaled airflow volume, a real-time inhaled airflow pressure, a real-time inhaled airflow volume, a real-time breathing minute volume, a real-time SpO₂ level, and a real-time HRV level.

The at least one air parameter sensor [112f] of the control unit determines one or more real-time physical properties of one or more of ambient air and enriched air within the enrichment airflow pathway [102c]. The one or more real-time physical properties includes, but is not limited to, a real-time air pressure, a real-time humidity content, a real-time temperature, and/or the like, of the one or more of ambient air and enriched air within the enrichment airflow pathway [102c].

The Input-output (I/O) unit [114] is a display unit provided to one or more of receive input from a user, and/or display values to the user. In one embodiment, the I/O unit [114] is adapted to directly receive a user input corresponding to the defined CO₂ concentration and the defined O₂ concentration. In another embodiment, the I/O unit [114] is adapted to receive a user input corresponding to a goal physiological parameter of the user. ‘Goal’ herein refers to the targeted (to be achieved) parameters. The goal physiological parameter of the user may include, but is not limited to, a goal heart rate, a goal exhaled airflow pressure, a goal exhaled airflow volume, a goal inhaled airflow pressure, a goal inhaled airflow volume, a goal breathing minute volume, a goal SpO₂ level, and a goal HRV level. In the embodiment of the I/O unit [114] receiving the goal physiological parameters from the user, the control unit [112] deploy an algorithm and/or artificial intelligence unit, to perform calculations on the goal physiological parameters to determine a calculated CO₂ concentration and a calculated O₂ concentration, which correspond to the defined CO₂ concentration and the defined O₂ concentration.

The controller [112g] is in communication with each of the at least one CO₂ actuator [112a, 112b], the O₂ actuator [112c], the CO₂ sensor [112d], the O₂ sensor [112e], the one or more physiological parameter sensors, and the at least one air parameter sensor [112f]. The controller [112g] is adapted to: receive the CO₂ signals corresponding to CO₂ concentration in the enriched air from the CO₂ sensor [112d]; receive O₂ signals corresponding to O₂ concentration in the enriched air from the O₂ sensor [112e]; receive the defined CO₂ concentration and the defined O₂ concentration received from the user at the I/O unit [114]; compare the CO₂ concentration and the O₂ concentration in the enriched air with the defined CO₂ concentration and the defined O₂ concentration; and correspondingly control the at least one CO₂ actuator [112a, 112b] and the O₂ actuator [112c], for controlled manipulation of the at least one CO₂ modulation unit [106, 108] and the O₂ modulation unit [110]. The controlled manipulation of the at least one CO₂ modulation unit [106, 108] and the O₂ modulation unit [110] adjust the CO₂ concentration and the O₂ concentration in the enriched air disposed within the enrichment airflow pathway [102c] at least substantially equivalent to the defined CO₂ concentration and the O₂ concentration. Examples of the controller [112g] may include, any of, an 8081 microcontroller, an 8085 microcontroller, an 8051 microcontroller, a microprocessor, and/or the like.

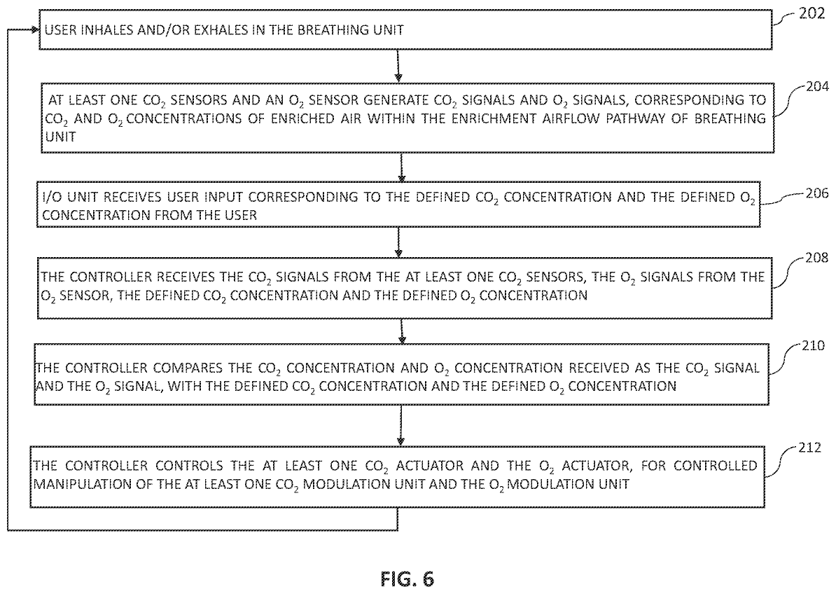

FIG. 6 shows a flowchart of a method of controlling the breathing unit [102] of the breath training apparatus [100], in accordance with the concepts of the first embodiment of the present disclosure. The method initiates at step [202].

At step [202], a user initiates inhalation and/or exhalation. Such step includes various sub-steps as defined herein. At a first sub-step, the user exhales an exhaled air in at least one exhalation pass, which is received through an air inlet of the breathing unit [102], into the enrichment airflow pathway [102c] of the breathing unit [102]. Thereafter, at a second sub-step, the enrichment airflow pathway [102c] of the breathing unit [102] enables, at least a portion of air in the enrichment airflow pathway [102c] to vent through the air outlet [102b], such that the remaining air in the enrichment airflow pathway [102c] mixes with the exhaled air to be enriched in CO₂ concentration. Thereafter, at a third sub-step, the user inhales the enriched air in the enrichment airflow pathway [102c] of the breathing unit [102] in subsequent inhalation pass, which is released through the air inlet [102a] for inhalation to the user. Thereafter, the method proceeds to step [204].

At step [204], the at least one CO₂ sensors [112d] generate CO₂ signals, corresponding to CO₂ concentration of the enriched air within the enrichment airflow pathway [102c] of the breathing unit [102]. Concurrently, the O₂ sensor [112e] generate O₂ signals, corresponding to O₂ concentration of the enriched air within the enrichment airflow pathway [102c] of the breathing unit [102]. The method then proceeds to step [206].

At step [206], the I/O unit receives a user input corresponding to the defined CO₂ concentration and the defined O2 concentration from the user. The method then proceeds to step [208].

At step [208], the controller [112g] receives: the CO₂ signals from the at least one CO₂ sensors [112d], the O₂ signals from the O₂ sensor [112e], and the defined CO₂ concentration and the defined O2 concentration from the I/O unit. The method then proceeds to step [210].

At step [210], the controller [112g] compares the CO₂ concentration and O₂ concentration received as the CO₂ signal and the O₂ signal, with the defined CO₂ concentration and the defined O₂ concentration. The method then proceeds to step [212].

At step [212], the controller controls the at least one CO₂ actuator [112a, 112b] and the O₂ actuator [112c], for controlled manipulation of the at least one CO₂ modulation unit [106, 108] and the O₂ modulation unit [110]. The controlled manipulation of the at least one CO₂ modulation unit [106, 108] adjust the CO₂ concentration and the O₂ concentration of the enriched air within the enrichment airflow pathway [102c] at least substantially equivalent to the defined CO₂ concentration and the defined CO₂ concentration, respectively.

Various advantages of the breath training apparatus [100], as disclosed in the present disclosure, are useful and beneficial used for breath trainings/exercises. For example, the breath training apparatus [100], as disclosed in the present disclosure, includes a large diameter (or large width) cuboidal housing unit (avoiding small orifices), for receiving ambient air and exhaled air, which are mixed together for providing inhalation air with increased CO₂ concentration and increased O₂ concentration. Usage of such large diameter (or large width) cuboidal-shaped housing unit [102d] enables relatively easy inhalation and exhalation of the user by reducing air resistance, during breath training exercises. Additionally, this increases relatively natural simulation of breathing, during breath training exercises. Moreover, the control unit [112] for the breathing unit [102] employs the CO₂ sensor [112d], the at least one CO₂ actuator [112a, 112b], and the controller [112g], such that the controller [112g] suitably manipulates the at least one CO₂ actuator [112a, 112b] based on signals from the CO₂ sensor[112d], to adjust the CO₂ concentration within the enriched air in the enrichment airflow pathway [102c]. As the control unit [112] operates on an electrically controlled units, it provides precise control of the CO₂ concentration within the inhalation air. Moreover, the housing unit [102d] of the breathing unit [102], as part of the disclosed breath training apparatus [100], has a volume in a range of 2.5 to liters, preferably in a range of 5 to 15 liters. This volume capacity is equal to or substantially above the vital capacity of the user. Accordingly, the breath training apparatus [100], as disclosed in the present disclosure, is adaptable to be adjusted to a varied amount of the CO₂ concentration within the inhalation air available for breathing during inhalation pass, based on user's requirements and user's vital capacity.

While the preferred embodiments of the present invention have been described hereinabove, it should be understood that various changes, adaptations, and modifications may be made therein without departing from the spirit of the invention and the scope of the appended claims. It will be obvious to a person skilled in the art that the present invention may be embodied in other specific forms without departing from its spirit or essential characteristics. The described embodiments are to be considered in all respects only as illustrative and not restrictive.

### LIST OF COMPONENTS

100—Breath Training Apparatus
102—Breathing Unit
102a—Air Inlet
102b—Air outlet
102c—Enrichment Pathway
102d—Housing Unit
104—Mask Unit
106—First CO₂ modulation unit
108—Second CO₂ modulation unit
108a—Secondary Housing Units
110—O₂ modulation unit
110a—O₂ connection point
110b—O₂ vent hole
110c—O₂ entry hole
112—Control Unit
112a—First CO₂ actuator
112b—Second CO₂ actuator
112c—O₂ actuator
112d—CO₂ sensor
112e—O₂ sensor
112f—Air Parameter Sensor
112g—Controller

1. A breath training apparatus, comprising:
a breathing unit defining an air inlet, an air outlet, and an enrichment airflow pathway therebetween, wherein the breathing unit is adapted to:
receive exhaled air from a user in at least one exhalation pass through the air inlet;
enable a portion of air in the enrichment airflow pathway to vent through the air outlet, such that the remaining air in the enrichment airflow pathway mixes with the exhaled air to be enriched in CO₂ concentration, and release at least a portion of the enriched air through the air inlet for inhalation in subsequent inhalation pass to the user;
at least one CO₂ modulation unit coupled to the breathing unit for modulating the CO₂ concentration in the enriched air, such that a controlled manipulation of the at least one CO₂ modulation unit corresponds to a control of the CO₂ concentration within the enrichment airflow pathway of the breathing unit; and
a control unit, comprising:
at least one CO₂ actuator for controllably manipulating the at least one CO₂ modulation unit to control the CO₂ concentration within the enrichment airflow pathway of the breathing unit; and
a CO₂ sensor for generating signals corresponding to the CO₂ concentration of the enriched air within the enrichment airflow pathway of the breathing unit; and
a controller for receiving the signals corresponding to the CO₂ concentration within the enrichment airflow pathway from the CO₂ sensor, comparing the CO₂ concentration with a defined CO₂ concentration, and correspondingly controlling the at least one CO₂ actuator for the controlled manipulation of the at least one CO₂ modulation unit, such that the controlled manipulation of the at least one CO₂ modulation unit adjusts the CO₂ concentration within the enrichment airflow pathway at least substantially equivalent to the defined CO₂ concentration.

2. The breath training apparatus of claim 1, further comprising an O₂ supply unit fluidly communicating with the enrichment airflow pathway of the breathing unit for supplying O₂ to the enriched air within the enrichment airflow pathway.

3. (canceled)

4. (canceled)

5. (canceled)

6. The breath training apparatus of claim 1, further comprising an O₂ sensor for measuring and generating O₂ signals corresponding to O₂ concentration of the enriched air within the enrichment airflow pathway of the breathing unit.

7. (canceled)

8. The breath training apparatus of claim 1, wherein the breathing unit has an enclosed housing unit defining the enrichment airflow pathway therein, such that the enrichment airflow pathway has a predefined trajectory, and wherein the predefined trajectory is one of: a straight trajectory defined between the air inlet and the air outlet, or a curved trajectory with at least one turn between the air inlet and the air outlet.

9. (canceled)

10. (canceled)

11. (canceled)

12. The breath training apparatus of claim 1, wherein one of the at least one CO₂ modulation unit is a closable opening positioned proximal to the air inlet, for enabling controlled fluid communication of the enrichment airflow pathway with ambient air in external environment corresponding to the controlled manipulation of the first one of the at least one CO₂ modulation unit for the control of the CO₂ concentration within the enrichment airflow pathway of the breathing unit.

13. The breath training apparatus of claim 1, wherein one of the at least one CO₂ modulation unit is a secondary housing unit telescopically extending from a housing unit of the breathing unit, such that a controlled telescopic extension and/or retraction of the secondary housing unit relative to the housing unit enlarges and/or contracts the enrichment airflow pathway, corresponding to the controlled manipulation of the one of the at least one CO₂ modulation unit for the control of the CO₂ concentration within the enrichment airflow pathway of the breathing unit.

14. (canceled)

15. (canceled)

16. The breath training apparatus of claim 1, further comprising one or more real-time physiological sensors for determining one or more real-time physiological parameters of the user wherein the one or more real-time physiological parameters comprise at least one of: a heart rate, an exhaled airflow pressure, an exhaled airflow volume, an inhaled airflow pressure, an inhaled airflow volume, a breathing minute volume, a SpO₂ level, and a heart rate variability (HRV) level.

17. The breath training apparatus of claim 1, further comprising at least one real-time air parameter sensor for determining one or more real-time physical properties of one or more of ambient air and the enriched air within the enrichment airflow pathway.

18. (canceled)

19. The breath training apparatus of claim 1, further comprising an I/O unit for receiving a user input corresponding to the defined CO₂ concentration and a defined O₂ concentration, and displaying in real-time the CO₂ concentration, O₂ concentration, one or more real-time physiological parameters related to the user, and one or more physical properties of ambient air.

20. (canceled)

21. (canceled)

22. (canceled)

23. (canceled)

24. (canceled)

25. The breath training apparatus of claim 1, wherein the air inlet is fluidly connected to a mask unit to receive the exhaled air from the user and supply the enriched air to the user.

26. The breath training apparatus of claim 1, wherein the air inlet is fluidly connected to an enclosed tent unit or a cocoon unit housing the user, to receive the exhaled air from the user and supply the enriched air to the user.

27. The breath training apparatus of claim 1, wherein the air inlet is fluidly connected to a mask unit housing at least a head of the user, to receive the exhaled air from the user and supply the enriched air to the user.

28. (canceled)

29. The breath training apparatus of claim 1, wherein the CO₂ sensor is one of: an air pressure sensor or an airflow sensor.

30. (canceled)

31. A method for automatically controlling a breath training apparatus the method comprising:
receiving, by an air inlet of a breathing unit, an exhaled air from a user in at least one exhalation pass;
enabling, by the breathing unit, at least a portion of air in an enrichment airflow pathway of the breathing unit to vent through an air outlet of the breathing unit, such that the remaining air in the enrichment airflow pathway mixes with the exhaled air to be enriched in CO₂ concentration;
releasing, by the air inlet of the breathing unit, at least a portion of the enriched air for inhalation in subsequent inhalation pass to the user;
generating signals, by a CO₂ sensor, corresponding to the CO₂ concentration of the enriched air within the enrichment airflow pathway of the breathing unit;
receiving, by a controller, the signals;
receiving, by an I/O unit, a defined CO₂ concentration from the user;
comparing, by the controller, the CO₂ concentration based on the signals with the defined CO₂ concentration; and
controlling, by the controller, at least one CO₂ actuator for controlled manipulation of at least one CO₂ modulation unit, such that the controlled manipulation of the at least one CO₂ modulation unit adjusts the CO₂ concentration within the enrichment airflow pathway at least substantially equivalent to the defined CO₂ concentration.

32. The method of claim 31, further comprising the step of supplying, by an O₂ supply unit, O₂ to the enriched air within the enrichment airflow pathway.

33. (canceled)
